(19) United States
(12) Patent Application Publication
(10) Pub. No.: US 2020/0090810 A1
(43) Pub. Date: Mar. 19, 2020

(54) MEDICAL INFORMATION PROCESSING APPARATUS, METHOD AND SYSTEM

(71) Applicant: Canon Medical Systems Corporation, Otawara-shi (JP)

(72) Inventor: Takuya Sakaguchi, Utsunomiya (JP)

(73) Assignee: Canon Medical Systems Corporation, Otawara-shi (JP)

(21) Appl. No.: 16/561,851

(22) Filed: Sep. 5, 2019

(30) Foreign Application Priority Data
Sep. 6, 2018 (JP) 2018-167029
Sep. 3, 2019 (JP) 2019-160574

(51) Int. Cl. G16H 50/20; G16H 50/30; G06N 20/00; G16H 10/60

(52) U.S. Cl. CPC G16H 50/20; G16H 10/60; G06N 20/00; G16H 50/30

(57) ABSTRACT

According to one embodiment, a medical information processing apparatus includes processing circuitry. The processing circuitry acquires a plurality of processed medical signals regarding a subject by performing at least one of different imaging techniques and different signal processing operations. The processing circuitry outputs a diagnosis result by applying a trained model to the plurality of processed medical signals.

### CROSS-REFERENCE TO RELATED APPLICATIONS

This application is based upon and claims the benefit of priority from the prior Japanese Patent Application No. 2018-167029, filed Sep. 6, 2018, and No. 2019-160574, filed Sep. 3, 2019, the entire contents of all of which are incorporated herein by reference.

### FIELD

Embodiments described herein relate generally to a medical information processing apparatus, a medical information processing method, and a medical information processing system.

### BACKGROUND

A system called computer-aided diagnosis (CAD), which analyzes an image taken as the input, and outputs a result of diagnosis of the image or assistance information on the diagnosis, has been used. However, the stability of the output result may be dependent on the quality of the input image, or the result of diagnosis may not be sufficiently reliable.

### BRIEF DESCRIPTION OF THE DRAWINGS

FIG. 1 is a block diagram showing a configuration example of a hospital information system in which a medical information processing apparatus is provided.

FIG. 2 is a block diagram showing a configuration example of the medical information processing apparatus.

FIG. 3 is a block diagram showing an example of a training system that generates a trained model.

FIG. 4 is a conceptual diagram showing a typical configuration of a multi-layered network.

FIG. 5 is a conceptual diagram showing a data flow of a first processing example according to the first embodiment.

FIG. 6 is a conceptual diagram showing a data flow of a second processing example according to the first embodiment.

FIG. 7 is a conceptual diagram showing a data flow of a third processing example according to the first embodiment.

FIG. 8 is a conceptual diagram showing a data flow of a first processing example according to the second embodiment.

FIG. 9 is a conceptual diagram showing a data flow of a second processing example according to the second embodiment.

### DETAILED DESCRIPTION

In general, according to one embodiment, a medical information processing apparatus includes processing circuitry. The processing circuitry acquires a plurality of processed medical signals regarding a subject by performing at least one of different imaging techniques and different signal processing operations. The processing circuitry outputs a diagnosis result by applying a trained model to the plurality of processed medical signals.

Hereinafter, a medical information processing apparatus, a medical information processing method, and a medical information processing system according to the embodiment will be described, with reference to the accompanying drawings. In the embodiments described below, elements assigned with the same reference symbols are assumed to perform the same operations, and redundant descriptions thereof will be suitably omitted.

Embodiments will be described below with reference to the accompanying drawings.

### First Embodiment

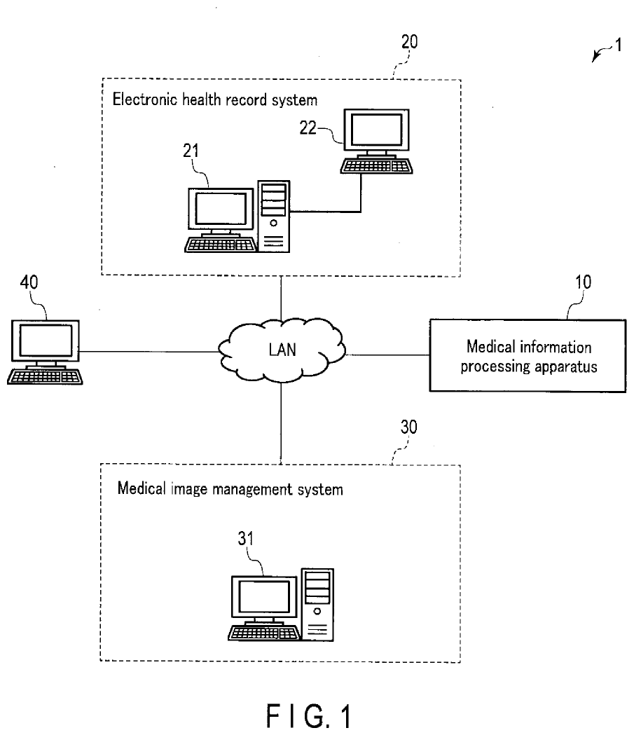

FIG. 1 is a block diagram showing a configuration example of a hospital information system 1 in which a medical information processing apparatus 10 according to the first embodiment is provided. The hospital information system 1 shown in FIG. 1 includes a medical information processing apparatus 10, an electronic health record system 20, a medical image management system (Picture Archiving and Communication System (PACS)) 30, and a communication terminal 40. The medical information processing apparatus 10, the electronic health record system 20, the medical image management system 30, and the communication terminal 40 are connected via an intra-hospital network such as a local area network (LAN) in a communicatory manner enabling data communications. The connection to an intra-hospital network may be either wired or wireless. As long as the security is ensured, the connection need not necessarily be made to an intra-hospital network. The connection may be made to, for example, a public communication line, such as the Internet, via a virtual private network (VPN) or the like.

The electronic health record system 20 is a system that stores electronic health record data including diagnosis and treatment information, patient information, etc. It also manages the stored electronic health record data. The diagnosis and treatment information includes information on the electronic health record, such as information on findings, disease name information, vital sign information, test stage information, and information on details of the treatment. The patient information includes, for example, a patient ID, a patient's name, gender, age, etc.

The electronic health record system 20 includes, for example, a server apparatus 21 and a communication terminal 22. The server apparatus 21 and the communication terminal 22 are connected via an intra-hospital network in a communicatory manner. In the electronic health record system 20, the server apparatus 21 stores diagnosis and treatment information, patient information, etc., and manages the stored diagnosis and treatment information, patient information, etc. In response to an output request, the server apparatus 21 outputs, for example, the stored diagnosis and treatment information, patient information, etc. to the requester.

FIG. 1 shows, as an example, the case where the server apparatus 21 is the only server included in the electronic health record system 20; however, the configuration is not limited thereto. A plurality of server apparatuses 21 may be provided as needed. For example, the server apparatus 21 may be provided for each item of information to be managed.

The communication terminal 22 is a terminal for medical staff, such as a doctor, to access the server apparatus 21. Specifically, for example, the communication terminal 22 is operated by medical staff, and requests the server apparatus 21 for information stored in the server apparatus 21.

The medical image management system 30 is a system that stores medical image data and manages the stored medical image data. The medical image management system 30 includes, for example, a server apparatus 31. In the medical image management system 30, the server apparatus 31 stores medical image data converted in accordance with, for example, the digital imaging and communication medicine (DICOM) standard, and manages the stored medical image data. The server apparatus 31 transmits, in response to a browsing request, for example, the stored medical image data to the requester.

FIG. 1 shows, as an example, the case where the server apparatus 31 is the only server included in the medical image management system 30; however, the configuration is not limited thereto. A plurality of server apparatuses 31 may be provided as needed.

The communication terminal 40 is a terminal for medical staff, such as a doctor, to access a system, an apparatus, etc. connected to the LAN.

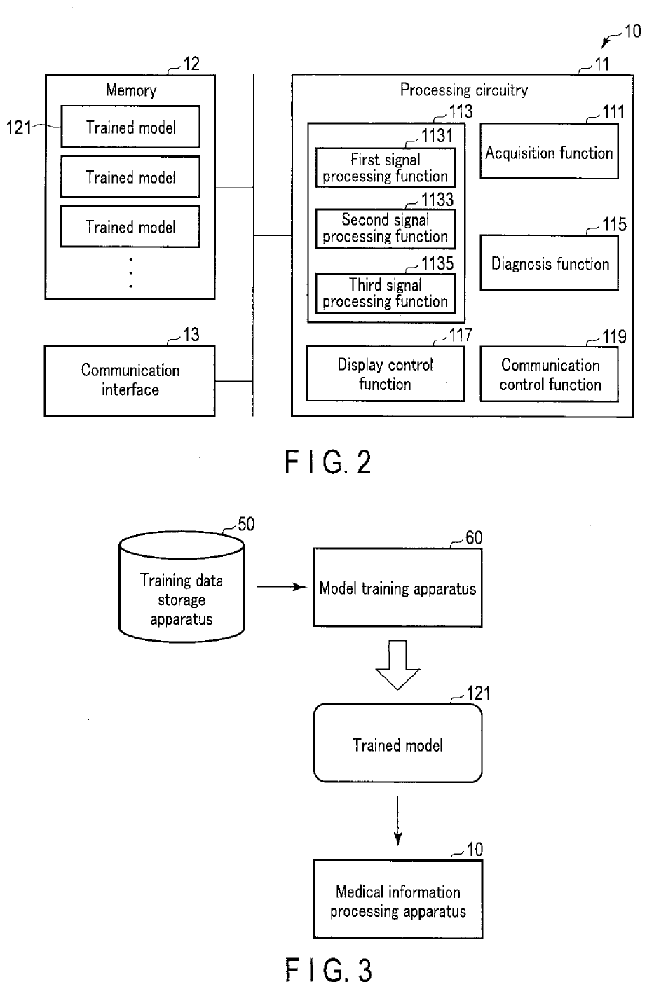

The medical information processing apparatus 10 is an apparatus that assists an operator, such as a doctor, in the diagnosis of a patient. FIG. 2 is a block diagram illustrating an example of a functional configuration of the medical information processing apparatus 10 shown in FIG. 1. The medical information processing apparatus 10 shown in FIG. 2 includes processing circuitry 11, a memory 12, and a communication interface 13. The processing circuitry 11, the memory 12, and the communication interface 13 are, for example, connected to one another via a bus in a communicatory manner.

The medical information processing apparatus 10 may be a computer installed in a medical image diagnosis apparatus in which a medical imaging apparatus is installed, a computer connected to the medical image diagnosis apparatus via a cable, a network, etc., in a communicatory manner, or a computer independent from the medical image diagnosis apparatus.

The processing circuitry 11 is a processor that functions as a nerve center of the medical information processing apparatus 10. The processing circuitry 11 executes a program stored in, for example, the memory 12, and implements the function corresponding to the executed program.

The processing circuitry 11 shown in FIG. 2 executes a diagnosis assistance program stored in the memory 12, thereby implementing the function corresponding to the executed program. Through execution of the diagnosis assistance program, the processing circuitry 11 performs, for example, an acquisition function 111, a signal processing function 113, a diagnosis function 115, a display control function 117, and a communication control function 119. The functions 111 to 119 are not necessarily implemented by a single processing circuit. The processing circuitry may be configured by combining a plurality of independent processors that respectively execute programs to implement the functions 111 to 119.

Through performance of the acquisition function 111, the processing circuitry 11 acquires a medical signal obtained from a subject. The medical signal is assumed to be, for example, an image signal or raw data; however, it may be intermediate data acquired prior to image data generated by the subjection of raw data to some signal processing. A plurality of medical signals may be acquired as a plurality of processed medical signals by the performance of different imaging techniques.

The image based on the image signal is, for example, an ultrasound image, a computed tomography (CT) image, a magnetic resonance (MR) image, a positron-emission tomography (PET) image, or a single-photon emission computed tomography (SPECT) image. Examples of the raw data or intermediate data include, but are not limited to, k-space data acquired in the magnetic resonance imaging apparatus, projection data or sinogram data acquired by an X-ray CT apparatus, spectrum data acquired by a photon-counting CT apparatus, image data with different energy bands or image data acquired by material discrimination, echo data acquired by an ultrasound diagnostic apparatus, coincidence data or sinogram data acquired by a PET apparatus, projection data or sinogram data acquired by a SPECT apparatus, and an ECG waveform acquired by an electrocardiogram.

Through performance of the signal processing function 113, the processing circuitry 11 performs different signal processing operations on a medical signal, and generates a processed medical signal for each signal processing operation. The processing performed by the signal processing function 113 may be sequential processing, in which, for example, a processed medical signal obtained by a certain signal processing operation is transferred to and processed at a subsequent signal processing operation. Alternatively, the processing performed by the signal processing function 113 may be parallel processing, in which a medical signal is input to the signal processing function 113, and a plurality of signal processing operations are performed on the medical signal.

As a concrete example, let us assume, in the present embodiment, that the processing circuitry 11 performs three signal processing functions, namely, a first signal processing function 1131, a second signal processing function 1133, and a third signal processing function 1135. The configuration of the processing circuitry 11 is not limited thereto, and the processing circuitry 11 may perform four or more signal processing functions.

Through performance of the first signal processing function 1131, the processing circuitry 11 receives a medical signal, and performs a first signal processing operation on the received medical signal. In the present embodiment, let us assume that image reconstruction is performed as the first signal processing operation, and thereby a reconstructed image signal is generated as a processed medical signal. If the medical signal is projection data or sinogram data acquired by an X-ray CT apparatus, a reconstructed CT image signal is generated using Filtered Back Projection. If the medical signal is k-space data acquired by an MRI apparatus, a reconstructed MR image signal is generated by applying the Fourier transform to the k-space data.

Through performance of the second signal processing function 1133, the processing circuitry 11 receives a reconstructed image signal from a first signal processing function 1131, and performs a second signal processing operation. In the present embodiment, let us assume that filtering is performed on the reconstructed image signal as the second signal processing operation, and a filtered image signal is generated as a processed medical signal. Example filtering processes include noise reduction, edge enhancement, smoothing, and contrasting.

Through performance of the third signal processing function 1135, the processing circuitry 11 receives the filtered image signal from the second signal processing function 1133, and performs a third signal processing operation. In the present embodiment, let us assume that analytical processing is performed on the filtered image signal as the third signal processing operation, and an analyzed image signal is generated as a processed medical signal. Concrete examples of the analytical processing include segmentation of an organ, etc., and size measurement of a tumor, etc.

Since the processed medical signals generated by the signal processing function 113 can be used as training data during training, the acquisition function 111 and the signal processing function 113 may constitute part of the training data generation apparatus.

Through performance of the diagnosis function 115, the processing circuitry 11 outputs a diagnosis result by applying a trained model (e.g., a multi-layered network with parameters adjusted by machine learning) to the generated processed medical signals. Specifically, the reconstructed image signal, the filtered image signal, and the analyzed image signal are respectively received from the first signal processing function 1131, the second signal processing function 1133, and the third signal processing function 1135. Through performance of the diagnosis function 115, the processing circuitry 11 outputs a diagnosis result of the medical image by applying a trained model based on machine learning (to be described later) to the reconstructed image signal, the filtered image signal, and the analyzed image signal.

The diagnosis result is assumed to be, for example, a result of the process of determining whether a tumor is benign or malignant; however, the diagnosis result is not limited thereto. The diagnosis result may be a result of the process of determining whether a disease is benign or malignant, a result of the process of abnormality determination as to whether a disease is suspected or not, as well as a result of automatic extraction of a region such as an organ position, the type, the usage, and the dosage of medication. Alternatively, the diagnosis result may be a result of the process of determining whether or not a disease requires a treatment or operation, namely, a result of the process of determining whether an operation or a treatment other than an operation need be performed for a disease, or whether a wait-and-see approach should be taken. The diagnosis result may be a combination of the above-described items of information.

The processing circuitry 11 may output the diagnosis results as numerals (continuous values), or as classification information categorized as, for example, 1, 2, and 3 (or A, B and C). Examples of the categories include stages indicating the extent to which a cancer has developed.

As the diagnosis result, the processing circuitry 11 may output information indicating the position at which a cancer has been detected. For example, the processing circuitry 11 detects the position of a cancer in images acquired based on the processed medical signals obtained in different signal processing operations, and outputs the detected positions. In this case, if a cancer is detected at the same position in the images acquired based on the processed medical signals, the reliability of the diagnosis results will be further increased.

Through performance of the display control function 117, the processing circuitry 11 performs control in such a manner that the diagnosis result is displayed on, for example, a display (not illustrated). The processing circuitry 11 may perform control in such a manner that a medical signal and a processed medical signal (the reconstructed image, the filtered image, and the analyzed image in the example of the present embodiment) are displayed, together with the diagnosis result.

Through performance of the communication control function 119, the processing circuitry 11 controls communication between the medical information processing apparatus 10 and systems such as the medical image management system 30 and unillustrated medical imaging apparatuses (e.g., MRI apparatus and an X-ray CT apparatus) via a communication interface 13 and a LAN.

The communication interface 13 performs data communications between the electronic health record system 20, the medical image management system 30, and the communication terminal 40, connected via the intra-hospital network. The communication interface 13 performs data communications in conformity with, for example, a known preset standard. Communications with the electronic health record system 20 are performed in conformity with, for example, HL7. Communications with the medical image management system 30 are performed in conformity with, for example, DICOM.

The medical information processing apparatus 10 may include an input interface. The input interface receives various types of input operations from the user, converts a received input operation into an electrical signal, and outputs the electrical signal to the processing circuitry 11. The input interface is connected to, for example, an input device such as a mouse, a keyboard, a trackball, a switch, a button, a joystick, a touch pad, and a touch panel to which an instruction is input by a touch on its operation surface. The input device connected to the input interface may be an input device provided on another computer and connected via a network, etc.

The medical information processing apparatus 10 may include a display. The display displays various types of information, in accordance with an instruction from the processing circuitry 11. The display may display, for example, a graphical user interface (GUI) for receiving various types of operations from the user. For the display, for example, a cathode ray tube (CRT) display, a liquid crystal display, an organic electroluminesence display (GELD), a light-emitting diode (LED) display, a plasma display, and any other display can be suitably used.

The memory 12 is a storage device, such as a read-only memory (ROM), a random access memory (RAM), a hard disk drive (HOD), a solid-state drive (SSD), an integrated-circuit storage device, etc., which stores various types of information. The memory 12 may be, for example, a drive that reads and writes various kinds of information on a portable storage medium such as a CD-ROM drive, a DVD drive, or a flash memory, etc. The memory 12 need not necessarily be realized by a single storage device. The memory 12 may be realized by, for example, a plurality of storage devices. The memory 12 may be in another computer connected to the medical information processing apparatus 10 via a network. When the memory 12 is in another computer, the medical information processing apparatus 10 and another computer connected to the medical information processing apparatus 10 via a network constitute examples of the “medical information processing system”. The “medical information processing system” is not limited thereto, and it is only required that some of the functions of the medical information processing apparatus 10 are distributed over the medical information processing apparatus 10 and one or more other computers.

The memory 12 stores, for example, a diagnosis assistance program according to the present embodiment. The diagnosis assistance program may be stored in advance in, for example, the memory 12. Also, the diagnosis assistance program may be stored in a non-transitory storage medium and distributed, read from the non-transitory storage medium, and installed in the memory 12.

The memory 12 stores a trained model 121 as, for example, an identifier generated by machine learning. The trained model 121 is an example of a calculation model. In the present embodiment, the trained model 121 represents a model generated by training a machine learning model according to a model training program.

Next, a description will be made of a method of generating a trained model 121, with reference to FIG. 3.

FIG. 3 is a block diagram showing an example of a training system that generates a trained model 121. A medical information processing system shown in FIG. 3 includes the medical information processing apparatus 10, a training data storage apparatus 50, and a model training apparatus 60.

The training data storage apparatus 50 stores training data including a plurality of training samples. The training data storage apparatus 50 is, for example, a computer with a built-in large-capacity storage device. The training data storage apparatus 50 may be a large-capacity storage device connected to a computer via a cable or a communication network in a communicatory manner. As such a storage device, a hard disk drive (HDD), a solid state drive (SSD), or an integrated circuit storage device, etc., can be suitably adopted.

The model training apparatus 60 generates a trained model 121 by training a machine learning model based on training data stored in the training data storage apparatus 50, according to a model training program. Example machine learning algorithms according to the present embodiment include discriminant analysis, logistic regression, support-vector machines, neural networks, randomized trees, and subspace method. The model training apparatus 60 is a computer such as a workstation, including a processor such as a central processing unit (CPU), a graphics processing unit (CPU), etc.

The model training apparatus 60 and the training data storage apparatus 50 may be connected via a cable or a communication network in a communicatory manner. The training data storage apparatus 50 may be installed in the model training apparatus 60. In such cases, training data is supplied from the training data storage apparatus 50 to the model training apparatus 60. The model training apparatus 60 and the training data storage apparatus 50 need not be connected in a communicatory manner. In such a case, training data is supplied from the training data storage apparatus 50 to the model training apparatus 60, via a portable storage medium storing the training data thereon.

The medical information processing apparatus 10 and the model training apparatus 60 may be connected via a cable or a communication network in a communicatory manner. The medical information processing apparatus 10 and the model training apparatus 60 may be implemented on a single computer. In such cases, the trained model 121 generated by the model training apparatus 60 is supplied to the medical information processing apparatus 10. The medical information processing apparatus 10 and the model training apparatus 60 need not necessarily be connected in a communicatory manner. In such a case, the trained model 121 is supplied from the model training apparatus 60 to the medical information processing apparatus 10 via a portable storage medium, etc., storing the trained model 121 thereon.

The supply of the medical information processing apparatus 10 of the trained model 121 may be performed at any point in time after the manufacturing of the medical information processing apparatus 10. For example, the supply may be performed at a given point in time between the manufacturing and the installation of the medical information processing apparatus 10 in a medical facility, or at the time of maintenance. The supplied trained model 121 is stored in the memory 12 of the medical information processing apparatus 10.

The trained model 121 according to the present embodiment is, for example, a composite function with parameters obtained by synthesizing a plurality of functions, for outputting a diagnosis result such as disease name estimation, disease degree-of-malignancy estimation (determination as to whether a tumor is benign or malignant), prognostic prediction, etc. by taking a medical signal such as medical image data and non-image diagnosis and treatment information as the input.

A composite function with parameters is defined by a combination of a plurality of adjustable functions and parameters. The trained model 121 according to the present embodiment may be any composite function with parameters satisfying the above-described requirements.

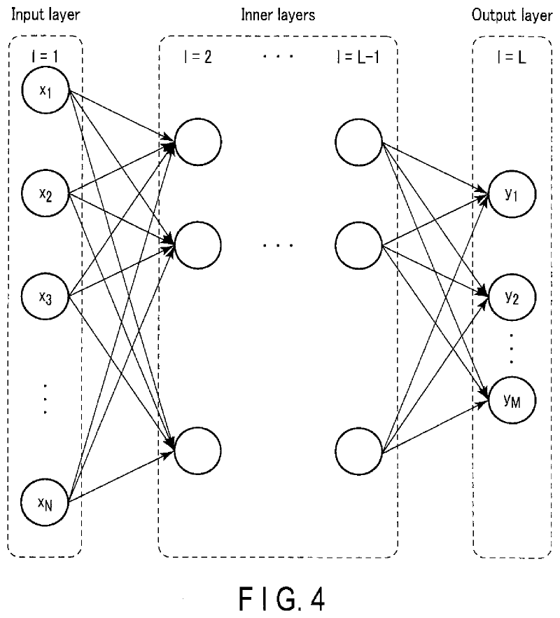

A typical configuration of a multi-layered network used as the trained model will be described with reference to the conceptual diagram of FIG. 4.

The multi-layered network is a network in which multiple layers are arranged in such a manner that only adjacent layers are connected, and information propagates in one direction, from the input layer side toward the output layer side. Let us assume that the multi-layered network according to the present embodiment comprises L layers, including an input layer (l=1), inner layers (l=2, 3, . . . , L−1), and an output layer (I=L), as shown in FIG. 4. A configuration of the multi-layered network will be described below as an example; however, the configuration is not limited thereto.

Assuming the number of units in the l-th layer as “I”, and expressing an input u^{(l)} to the l-th layer as in the formula (1-1) and an output z^{(l)} from the l-th layer as in the formula (1-2), the relationship between the input to the l-th layer and the output from the l-th layer can be expressed by the formula (1-3).

u^{(l)}=(u₁,u₂,u₃, . . . ,u₁) (1-1)

z^{(l)}=(z₁,z₂,z₃, . . . ,z₁) (1-2)

z^{(l)}=f(u^{(l)}) (1-3)

Here, the suffix “(l)” at the top right denotes the number of the layer. The “f(u)” in the formula (1-3) denotes an activation function, which may be selected, according to the purpose, from various functions, such as a logistic sigmoid function (logistic function), hyperbolic tangent function, a Rectified Linear Unit (ReLU), linear mapping, identity mapping, maxout functions, etc.

Assuming the number of units in the (l+1)-th layer as J, and the expression of a weighting matrix W^{(l+1)} between the l-th layer and the (l+1)-th layer as in the formula (2-1) and a bias b^{(l+1)} in the (l+1)-th layer as in the formula (2-2), an input u^{(l+1)} to the (l+1)-th layer and an output z^{(l+1)} from the (l+1)-th layer can be respectively expressed by the formulas (2-3) and (2-4).

$$\begin{matrix}{w}^{( l + 1 )} = ( \begin{matrix}{w}_{1 \; 1} & … & {w}_{1 l} \\ ⋮ & ⋱ & ⋮ \\ {w}_{J1} & … & {w}_{J l}\end{matrix} ) & ( 2 \text{-} 1 ) \\ {b}^{( l + 1 )} = ( {b}_{1} , {b}_{2} , {b}_{3 ,} … , {b}_{J} ) & ( 2 \text{-} 2 ) \\ {u}^{( l + 1 )} = {W}^{( l + 1 )} {z}^{( l )} + {b}^{( l + 1 )} & ( 2 \text{-} 3 ) \\ {z}^{( l + 1 )} = f ( {u}^{( l + 1 )} ) & ( 2 \text{-} 4 )\end{matrix}$$

In the multi-layered network according to the present embodiment, a medical signal represented by the formula (3-1) is input to the input layer (l=1). In the input layer, since the input data x is output as output data z^{(l)} without change, the relationship expressed by the formula (3-2) is satisfied.

x=(x₁,x₂,x₃, . . . ,x_{N}) (3-1)

z⁽¹⁾=x (3-2)

Assuming that the medical signal input to the input layer is referred to as “input medical signal”, various approaches may be selected, according to the purpose, as the input medical signal x. Typical examples will be listed below.

(1) An approach in which the input medical signal x is assumed to be a single item of image data, and each component xₚ (p=1, 2, . . . , N) is defined as a value (pixel or voxel value) representing one of the positions constituting the single item of image data.

(2) An approach in which the input medical signal x is assumed to be M items of image data (e.g., a plurality of items of image data with different imaging conditions), and a range of input units is assigned to each item of image data in the input layer, where each component xₚ is classified as the first item of image data if 1≤p≤q, as the second item of image data if q+1≤p≤r, and as the third item of image data if r+1≤p≤s.

(3) An approach in which the input medical signal x is assumed to be M items of image data, and each component xₚ is defined as a column vector consisting of a column of values (pixel values or voxel values) representing the respective positions of one item of image data.

(4) An approach in which one of the approaches (1)-(3), for example, is adopted, using raw data such as k-space data and projection data as the input medical signal x.

(5) An approach in which one of the approaches (1)-(3), for example, is adopted, using image data or raw data subjected to convolutional processing as the input medical signal x.

The outputs z⁽²⁾, . . . z^{(L-1)} of the respective inner layers (l=2, 3, . . . , L−1) subsequent to the input layer can be calculated by sequentially performing the calculations in accordance with the formulas (2-3) and (2-4).

The output z^{(L)} of the output layer (i.e., the L-th layer) is expressed as in the formula (4-1). The multi-layered network according to the present embodiment is a feedforward network in which image data x input to the input layer propagates from the input layer side toward the output layer side, via paths connecting only adjacent layers. Such a feedforward network can be expressed as a composite function as in formula (4-2).

$$\begin{matrix}{z}^{( L )} \text{:} y = {z}^{( L )} & ( 4 \text{-1} ) \\ \begin{matrix}y ( x ) = f ( {u}^{( L )} ) \\ = f ( {W}^{( L )} {z}^{( L - 1 )} + {b}^{( L )} ) \\ = f ( {W}^{( L )} f ( {W}^{( L - 1 )} {z}^{( L - 2 )} + {b}^{( L - 1 )} ) + {b}^{( L )} ) \\ = f ( {W}^{( L )} f ( {W}^{( L - 1 )} f ( … f ( {W}^{( l )} {z}^{( l - 1 )} + {b}^{( l )} ) … ) ) + {b}^{( L )} )\end{matrix} & ( 4 \text{-} 2 )\end{matrix}$$

By applying the formulas (2-3) and (2-4), the composite function defined by the formula (4-2) is defined as the combination of linear relationships of the layers using the weighting matrix W^{(l+1)}, non-linear relationships (or linear relationships) of the layers using the activation function f(u^{(l+1)}), and a bias b^{(l+1)}. In particular, the weighting matrix W^{(l+1)} and the bias b^{(l+1)} are referred to as “parameters p” of the network. The composite function defined by the formula (4-2) changes its functional form according to how the parameters p are selected. Accordingly, the multi-layered network according to the present embodiment can be defined as a function that allows the output layer to output a preferable result y, by suitably selecting the parameters p constituting the formula (4-2).

To suitably select the parameters p, training is performed using training data and an error function. Assuming that the desirable output (correct output) for the input xₙ is dₙ, the training data is a set D (n=1, . . . , S) of training samples (xₙ, dₙ) as expressed in formula (5-1).

(xₙ,dₙ) (5-1)

D={(x₁,d₁), . . . ,(x_{S},d_{S})} (5-2)

The error function is a function representing closeness between the training data dₙ and the output from the multi-layered network to which xₙ is input. Representative examples of the error function include the square error function, the maximum likelihood estimation function, the cross entropy function, etc. The type of function that should be selected as the error function depends on the problem being solved by the multi-layered network (e.g., a regression problem, a binary problem, a multi-class classification problem, etc.).

Let the error function be denoted by “E(p)”, and let the error function calculated using only one training sample (xₙ, dₙ) be denoted by “Eₙ(p)”. The current parameter p^{(t)} is updated to a new parameter p^{(t+1)} in accordance with the formula (6-1), which uses a gradient vector of the error function E(p), if the technique of gradient descent is adopted, and in accordance with the formula (6-3), which uses a gradient vector of the error function Eₙ(p), if the technique of stochastic gradient descent is adopted.

$$\begin{matrix}{p}^{( t + 1 )} = {p}^{( t )} - ɛ ∇ E ( {p}^{( t )} ) & ( 6 \text{-1} ) \\ ∇ E ( {p}^{( t )} ) ≡ \frac{\partial E}{\partial {p}^{( t )}} = [ \frac{\partial E}{\partial {p}_{1}^{( t )}} , … \frac{\partial E}{\partial {p}_{M}^{( t )}} ] & ( 6 \text{-} 2 ) \\ {p}^{( t + 1 )} = {p}^{( t )} - ɛ ∇ {E}_{n} ( {p}^{( t )} ) & ( 6 \text{-} 3 ) \\ ∇ {E}_{n ( {p}^{( t )} )} ≡ \frac{\partial {E}_{n}}{\partial {p}^{( t )}} = [ \frac{\partial {E}_{n}}{\partial {p}_{1}^{( t )}} , … \frac{\partial {E}_{n}}{\partial {p}_{M}^{( t )}} ] & ( 6 \text{-} 4 )\end{matrix}$$

Here, ε is a learning coefficient that determines the amount of update of the parameter p.

By slightly moving the current p to the negative gradient direction in accordance with the formula (6-1) or (6-3), and sequentially repeating the same, the parameters p that minimize the error function E(p) can be determined.

To calculate the formula (6-1) or (6-3), the gradient vector of E(p) expressed by the formula (6-2) or the gradient vector of Eₙ(p) expressed by the formula (6-4) need to be calculated. In the case where the error function is a square error function, as an example, the error function expressed by the formula (7-1) need to be differentiated with respect to the weight coefficient of each layer and the bias of each unit.

$$E ( {p}^{->} ) = \frac{1}{2} {\sum }_{n = 1}^{N} {‖ {d}_{n} - y ( {x}_{n} : p ) ‖}^{2} \qquad ( 7 \text{-} 1 )$$

Since the final output y is the composite function expressed by the formula (4-2), calculation of the gradient vector of E(p) or Eₙ(p) will be complex, and an enormous amount of calculation will be required.

Such inconveniences in gradient calculation can be resolved by the technique of backpropagation. For example, differentiation of the error function with respect to a weight wⱼᵢ^{(l)} of a path connecting the i-th unit of the (l-1)-th layer and the j-th unit of the l-th layer can be expressed as in the formula (8-1).

$$\frac{\partial {E}_{n}}{\partial {w}_{ji}^{( l )}} = \frac{\partial {E}_{n}}{\partial {u}_{j}^{( l )}} \frac{\partial {u}_{j}^{( l )}}{\partial {w}_{ji}^{( l )}} \qquad ( 8 \text{-} 1 )$$

The amount of change given to Eₙ by the input uⱼ⁽¹⁾ to the j-th unit of the l-th layer is caused merely by changing the input uₖ^{(l+1)} to each unit k of the (l+1)-th layer via the output zⱼ^{(l)} from the j-th unit. Thus, the first term of the right-hand side in the formula (8-1) can be expressed as in the formula (9-1), using the chain rule of differentiation.

$$\frac{\partial {E}_{n}}{\partial {u}_{j}^{( l )}} = {\sum }_{k} \frac{\partial {E}_{n}}{\partial {u}_{k}^{( l + 1 )}} \frac{\partial {u}_{k}^{( l + 1 )}}{\partial {u}_{j}^{( l )}} \qquad ( 9 \text{-} 1 )$$

Assuming that the left-hand side of the formula (9-1) is δⱼ^{(l)}, the formula (9-1) can be rewritten as in the formula (10-3), using the relationships of the formulae (10-1) and (10-2).

$$\begin{matrix}{u}_{k}^{( l + 1 )} = {\sum }_{j} {w}_{kj}^{( l + 1 )} {z}_{j}^{( l )} = {\sum }_{j} {w}_{kj}^{( l + 1 )} f ( {u}_{j}^{( l )} ) & ( 10 \text{-} 1 ) \\ \frac{\partial {u}_{k}^{( l + 1 )}}{\partial {u}_{j}^{( l )}} = {w}_{kj}^{( l + 1 )} \frac{\partial f ( {u}_{j}^{( l )} )}{\partial {u}_{j}^{( l )}} & ( 10 \text{-2} ) \\ {δ}_{j}^{( l )} = {\sum }_{k} {δ}_{k}^{l + 1} ( {w}_{kj}^{( l + 1 )} \frac{\partial f ( {u}_{j}^{( l )} )}{\partial {u}_{j}^{( l )}} ) & ( 10 \text{-} 3 )\end{matrix}$$

It can be seen, from the formula (10-3), that δⱼ^{(l)} of the left-hand side can be calculated by δₖ^{(l+1)} (k=1, 2, . . . ). That is, δⱼ^{(l)} of the l-th layer can be calculated if δₖ^{(l+1)} of the k-th unit of the subsequent (l+1)-th layer, located closer to the output layer by one, is given. Moreover, δₖ^{(l+1)} of the k-th unit in the (l+1)-th layer can be calculated if δₖ^{(l+2)} of the k-th unit of the subsequent (l+2)-th layer, located closer to the output layer by one, is given. By sequentially repeating such calculations, calculations can be performed up to the output layer.

If δₖ^{(L)} of the k-th unit in the L-th layer, namely, the output layer is found first, δₖ^{(l+1)} in a given layer can be calculated by repeating sequential calculations toward the previous layer side (i.e., the input layer side) (backpropagation) using the formula (10-3).

On the other hand, the second term of the right-hand side in the formula (8-1) can be calculated as in the formula (11-2), using the formula (11-1), which rewrites the formula (2-3) with respect to the components of the l-th layer.

$$\begin{matrix}{u}_{j}^{( l )} = {\sum }_{i} {w}_{ji}^{( l )} {z}_{i}^{( l - 1 )} & ( 11 \text{-1} ) \\ \frac{\partial {u}_{j}^{( l )}}{\partial {w}_{ji}^{( l )}} = {z}_{l}^{( l - 1 )} & ( 11 \text{-} 2 )\end{matrix}$$

Accordingly, differentiation of the error function with respect to a weight wⱼᵢ^{(l)} of a path connecting the i-th unit of the (l-1)-th layer and the j-th unit of the l-th layer can be expressed as in the formula (12-1), using the formula (8-1), δⱼ^{(l)} by the formula (10-3), and the formula (11-2).

$$\frac{\partial {E}_{n}}{\partial {w}_{ji}^{( l )}} = {δ}_{j}^{( l )} {z}_{i}^{( l - 1 )} \qquad ( 12 \text{-} 1 )$$

It can be seen, from the formula (12-1), that differentiation of the error function of the weight wⱼᵢ^{(l)} of a path connecting the i-th unit of the (l-1)-th layer and the j-th unit of the l-th layer is given by a product of δⱼ^{(l)} of the j-th unit and zᵢ^{(l-1)}, which is an output from the i-th unit. The Vu can be calculated by backpropagation using the formula (10-3), as described above, and the first value of the backpropagation, namely, δⱼ^{(L)} of the L-th layer, which is the output layer, can be calculated as in the formula (13-1).

$${δ}_{j}^{( L )} = \frac{\partial {E}_{n}}{\partial {u}_{j}^{( L )}} \qquad ( 13 \text{-} 1 )$$

Through the above-described procedure, training using a training sample (xₙ, dₙ) can be realized in the multi-layered network of the present embodiment. The gradient vector of the sum of errors E=ΣₙEₙ regarding a plurality of training samples can be acquired by repeating the above-described procedure in parallel for each training sample (xₙ, dₙ), and calculating the sum using the following formula (14-1):

$$\frac{\partial E}{\partial {w}_{ji}^{( l )}} = {\sum }_{n} \frac{\partial {E}_{n}}{\partial {w}_{ji}^{( l )}} \qquad ( 14 \text{-} 1 )$$

In the present embodiment, the training data can be a set of training samples containing a plurality of processed medical signals obtained by subjecting a medical signal to a plurality of signal processing operations as the input, and a diagnosis result regarding the degree of malignancy of the disease as the correct output. By performing machine learning based on the training data, the model training apparatus 60 generates a trained model that estimates a diagnosis result regarding the degree of malignancy of the disease based on the medical signal input.

The above-described trained model is assumed to be generated to detect the degree of malignancy of a tumor, irrelevant to a body part targeted for imaging; however, a trained model may be generated for each body part targeted for imaging, such as the heart, the liver, the lung, and the spine. For example, machine learning can be performed using training data containing a captured image of the heart captured and a processed image acquired by subjecting the captured image of the heart to an image processing operation as the input, and a diagnosis result as the correct output.

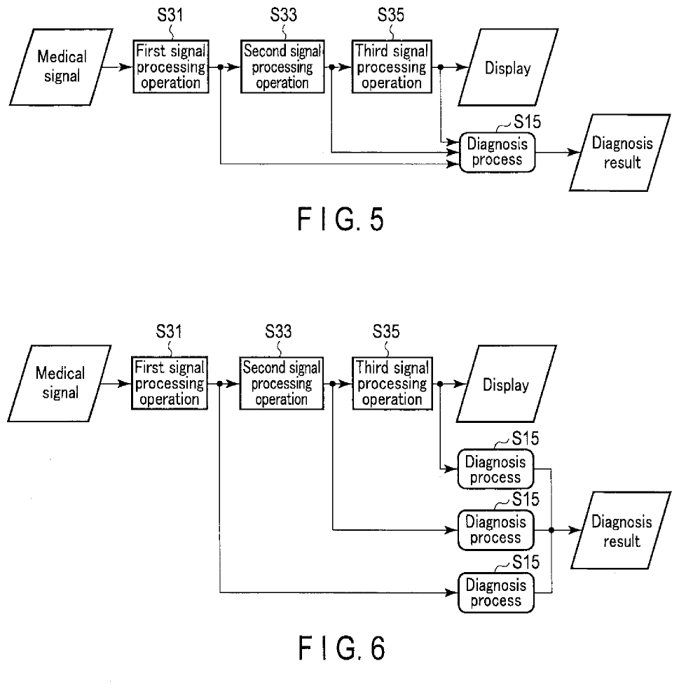

Next, a description will be given of a first processing example of the medical information processing apparatus 10 according to the first embodiment, with reference to FIG. 5.

FIG. 5 is a conceptual diagram showing a data flow according to a first processing example, from input of a medical signal to output of a diagnosis result.

Through performance of the first signal processing function 1131, the processing circuitry 11 performs a first signal processing operation S31 (image reconstruction in this case) on the input medical signal, and generates a reconstructed image signal. The reconstructed image signal is input to the second signal processing function 1133 and the diagnosis function 115.

Through performance of the second signal processing function 1133, the processing circuitry 11 performs a second signal processing operation S33 (filtering in this case) on the reconstructed image signal, and generates a filtered image signal. The filtered image signal is input to the third signal processing function 1135 and the diagnosis function 115.

Through performance of the third signal processing function 1135, the processing circuitry 11 performs a third signal processing operation S35 (a segmentation operation in this example) on the filtered image signal, and generates an analyzed image signal. The analyzed image signal generated by the third signal processing operation S35 is displayed on the display as an analyzed image via the display control function 117, and the analyzed image signal is input to the diagnosis function 115.

Through performance of the diagnosis function 115, the processing circuitry 11 outputs a diagnosis result by applying a trained model to the signals subjected to the respective signal processing operations, namely, the image signal, the filtered image signal, and the analyzed image signal. Specifically, a message such as “tumor is benign” or “tumor is malignant” is output as the diagnosis result, based on a pattern characteristic of three images.

In the present embodiment, by sequentially performing a plurality of signal processing operations such as signal processing and filtering on a single medical signal, a plurality of processed medical signals with different characteristics are generated.

Thereby, an analyzed image subjected to a plurality of stages of processing is displayed on the display screen, and a characteristic pattern that remains on the analyzed image, if any, is considered to remain in any course of signal processing. Thus, such a pattern can be extracted more easily through the processing by the diagnosis function 115.

That is, as compared to the case where, for example, only an image from which a characteristic pattern to be enhanced has been erased by strong smoothing filtering is input to a trained model, it is possible to leave a number of clues for extracting the characteristic pattern. This increases the possibility of high-precision extraction of a characteristic pattern to be extracted.

For the purpose of increasing the input data, the medical signal itself, in addition to the processed medical signal, may be used as the input of the trained model.

Machine learning-based training of a trained model used in the process according to the first processing example can be performed using, for example, training data containing a medical signal and three image signals subjected to a set of processing from the first signal processing operation S31 to the third signal processing operation S35 as the input, and a diagnosis result as to whether a tumor included in such images is benign or malignant as the correct output. Through machine learning based on deep learning, a model that outputs a diagnosis result indicating whether a tumor is benign or malignant based on a plurality of images is generated.

When a trained model is generated for each body part targeted for imaging and stored in the memory 12, as described above, the processing circuitry 11 may switch the corresponding trained model 121 according to the body part targeted for imaging of the acquired medical signal by performing the diagnosis function 115, and output a diagnosis result using the corresponding trained model 121.

Next, a description will be given of a second processing example of the medical information processing apparatus 10 according to the first embodiment, with reference to FIG. 6.

In the first processing example shown in FIG. 5, a single diagnosis result is output based on three processed medical signals; however, in FIG. 6, the processing circuitry 11 outputs a single diagnosis result for each of three processed medical signals, namely, three diagnosis results in total, by performing the diagnosis function 115.

Specifically, a reconstructed image signal obtained by the first signal processing operation S31, a filtered image signal obtained by the second signal processing operation S33, and an analyzed image signal obtained by the third signal processing operation S35 are individually input into a diagnosis process S15, which applies the same trained model. The processing circuitry 11, which performs the diagnosis function 115, may be configured to perform the diagnosis operation in parallel, by preparing three identical trained models in the diagnosis process S15. Herein, the “identical trained models” refer to trained models with the same parameters. Alternatively, the diagnosis process may be sequentially performed by allowing the processed medical signals (i.e., a reconstructed image signal, a filtered image signal, and an analyzed image signal) to be input to a single trained model one after another.

For the output of the diagnosis result, a final diagnosis result may be determined from the three diagnosis results generated, by majority voting. Specifically, when three diagnosis results, “benign”, “benign”, and “malignant” are output as a result of determination of the degree of malignancy of a tumor, the diagnosis result “tumor is benign” may be ultimately output.

The processing circuitry 11 may study track records of determination results respectively output based on the processed medical signals, such as the rate of adoption by majority voting, and skip part of the processing in such a manner that a diagnosis result from a processing path that outputs a diagnosis result with a low adoption rate (i.e., with a low quality) is not output. Alternatively, the processing circuitry 11 may perform weighting to reduce the weight on a low-quality diagnosis result.

If the processed medical signal in the second processing example is an image, the image displayed by the display control function 117 on the display is assumed to be an analyzed image that is a processed medical signal subjected to three signal processing operations. In this case, the image displayed on the display may be changed according to the contents of the diagnosis result of the output processed medical signal. If the diagnosis result based on the filtered image signal subjected to the second signal processing operation S33 includes the determination “tumor is malignant”, for example, the processing circuitry 11 outputs an image based on the filtered image signal on the display, using the display control function 117.

On-screen presentation of the filter image data which forms the basis for the determination of a tumor as malignant allows the user to visually recognize the specific image based on which a tumor is determined as malignant.

If the diagnosis result is clearly erroneous, for example, the user can enter the correct information, and thereby the processed medical signal and the correct information based on the users entry can be collected as training data, which can then be used to update the trained model.

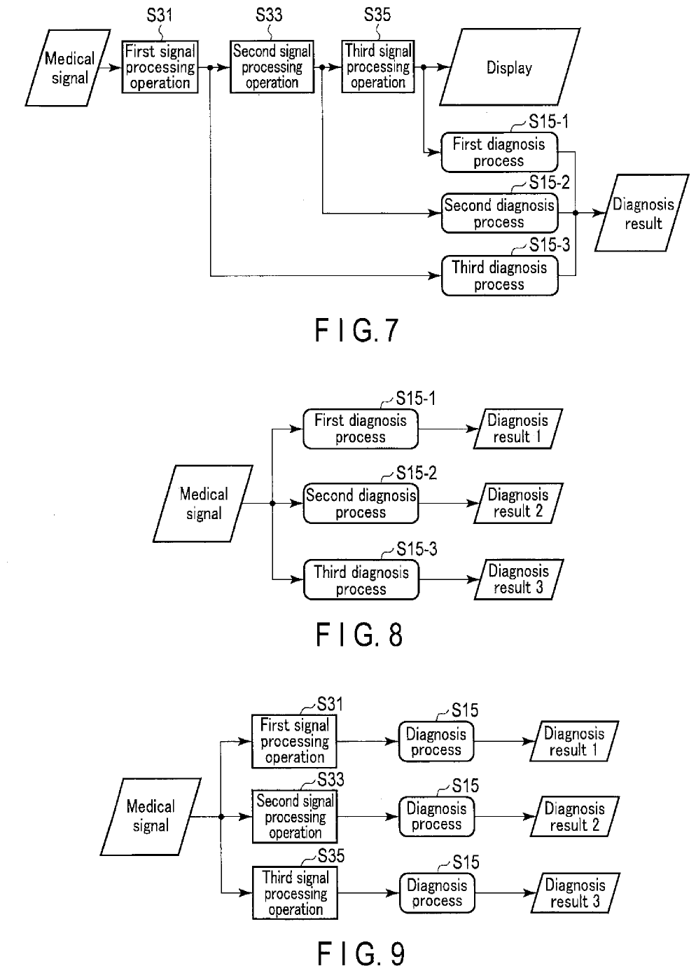

Next, a description will be given of a third processing example of the medical information processing apparatus 10 according to the first embodiment, with reference to FIG. 7.

In the second processing example shown in FIG. 6, a diagnosis process is performed on the processed medical signals by applying the same trained model thereto; however, in the third processing example shown in FIG. 7, the processing circuitry 11 performs a diagnosis process on three processed medical signals by applying different trained models thereto, through performance of the diagnosis function 115. The “different trained models” refer to trained models with different parameters.

For example, a trained model trained for a particular processed medical signal can be prepared according to the type of the processed medical signal. Specifically, a reconstructed image signal obtained in the first signal processing operation is subjected to a first diagnosis process S15-1, which applies a trained model optimized for image reconstruction. Similarly, a filtered image signal obtained in the second signal processing operation is subjected to a second diagnosis process S15-2, which applies a trained model optimized for filtering. The analyzed image signal output from the third signal processing function is subjected to a third diagnosis process S15-3, which applies a trained model optimized for a segmentation process.

Machine learning-based training of a trained model used in the diagnosis processes according to the third processing example can be performed using, for example, training data containing a medical signal and a processed medical signal subjected to the first signal processing operation S31 as the input, and a diagnosis result as to whether a tumor is benign or malignant as the correct output. Thereby, a fine-tuned trained model is generated for the processed medical signal subjected to the first signal processing operation S31.

By thus applying an optimized trained model for each of the processed medical signals, diagnosis of the processed medical signals can be performed with higher precision.

In addition to the above-described processes, the signal processing operations that will be described below may be performed as processes implemented by the first signal processing function, the second signal processing function, and the third signal processing function.

Example processes for deforming an image include resizing, rotation, trimming, binding (of signals at the detector level), and deformation. Three of the above-described processes may be respectively performed as the first signal processing operation, the second signal processing operation, and the third signal processing operation. Specifically, image resizing can be performed as the first signal processing operation, image rotation can be performed as the second signal processing operation, and image trimming can be performed as the third signal processing operation.

If the processed medical signals output from the first to third signal processing functions are different, a single operation may be repeated multiple times. In a concrete example, an image can be resized to 1024×1024 in the first signal processing operation, resized to 512×512 in the second signal processing operation, and resized to 256×256 in the third signal processing operation.

The processes that will be listed below can also be performed as each of the first to third signal processing operations.

Example processes for changing gray levels of an image include change of the window width and window level (WW/WL), gamma correction, dynamic range change, contrast conversion, logarithmic transformation, and bit conversion.

Example space filtering processes include mean filtering, median filtering, nose reduction filtering, and non-local mean filtering.

Example frequency conversion processes include high-pass filtering, low-pass filtering, and band-pass filtering, which passes one or more frequency bands.

Example sampling processes include upsampling and downsampling.

Example image correction processes include motion compensation, registration, segmentation, edge enhancement, thinning, dilation, and interpolation.

Example items that can be changed in image reconstruction include algorithms, reconstruction parameters, resolution, coefficients, slice thickness, the angle of view to be acquired (full scan, half scan, 70% scan, etc.), the sparse data acquisition position, and the type of super-resolution.

Example items that can be changed regarding the subject include the cardiac phase (time window) of data to be used and the respiratory phase. For example, variations may be provided between image data acquired at the time of the maximal inspiration and image data acquired at the maximal expiration. The items that can be changed regarding the subject may be set in advance of acquisition of a medical signal, as a different imaging technique.

Example items that can be changed regarding the X-ray CT apparatus include the tube voltage in the dual-energy technique, the reference material in decomposition, coefficients of subtraction, and the energy bins in photon counting. The items that can be changed regarding the X-ray CT apparatus may be set in advance of acquisition of a medical signal, as a different imaging technique.

Example items that can be changed for the deviation in the temporal direction include coefficients of a recursive filter, a sampling number, and data slightly shifted in the temporal direction. For the deviation in the temporal direction, frame addition or grid oscillation for acquiring images with no moiré patterns by oscillating anti-scatter grids may be performed.

Variations in data to be processed are caused according to, for example, whether data is processed as k-space data or as a sinogram. The other variations include a variation in compression processing, a variation in color conversion (e.g., from RGB to CMYK), etc.

The above-described processes can be suitably combined as signal processing operations, unless they are in the order that is not generally assumed.

For example, applying a mean filter prior to data selection at the maximum inspiratory phase is not generally assumed. Thus, the selection of data at the maximum inspiratory phase can be set as the first signal processing operation, and the application of a mean filter can be set as the second signal processing operation.

A combination of signal processing operations may be executed according to the case, by determining in advance what signal processing operations should be combined in what case on an empirical basis or according to the application of the device to be sold.

According to the first embodiment described above, a single medical signal is taken as the input, and the medical signal is subjected to signal processing operations to generate a plurality of processed medical signals, which are in turn taken as the input to generate a diagnosis result by applying a trained model thereto. Thus, even when the trained model has been trained on little data, and does not sufficiently correspond to the assumed input distribution (input width), it is possible to provide variations in data based on a medical signal, and to output a result based on different processed medical signals, at the time of use. Thus, unlike padding the input data at the time of training a model, it is possible to increase the diagnostic materials for the medical signal input to a trained model simply by preparing a single medical signal as the input on the user's side, and without preparing a plurality of images as the input, at the time of use.

In addition, since trained models can be prepared for the respective processed medical signals, scalable changes, such as changing only one trained model in accordance with a processed medical signal, can be performed, unlike a single trained model that includes multiple signal processing operations.

Consequently, it is possible to provide the user with a higher-precision diagnosis result, while significantly reducing the burden on the user, thus improving the reliability of the CAD-based diagnosis result.

### Second Embodiment

According to a second embodiment, let us assume that an output of the diagnosis result from a medical information processing apparatus 10 is used as, for example, a second opinion.

A description will be given of a first processing example of the medical information processing apparatus 10 according to the second embodiment, with reference to FIG. 8.

In the medical information processing apparatus 10 shown in FIG. 8, a medical signal is input to a diagnosis function 115. Through performance of the diagnosis function 115, processing circuitry 11 separately performs a first diagnosis process S15-1, a second diagnosis process S15-2, and a third diagnosis process S15-3 on the input medical signal. The signal input to the diagnosis function 115 is not limited to a medical signal, and may be a processed medical signal subjected to some signal processing, such as filtering. That is, it is only required that an identical signal be input to the first to third diagnosis processes S15.

The trained models used in the first diagnosis process S15-1 to the third diagnosis process S15-3 have been separately trained.

For acquisition of different trained models, for example, trained models trained at different hospitals, for example, may be used. Since the manufacturer, the model number, and the technician of the imaging device differ from hospital to hospital, different trained models are obtained if training is performed based on medical signals of the same body part targeted for imaging. Different trained models are obtained if images are captured at different periods of time, such as an image captured during the daytime and an image captured during the nighttime.

Three diagnosis results, which are results of the first diagnosis process S15-1, the second diagnosis process S15-2, and the third diagnosis process S15-3 are output via the diagnosis function 115. The three output diagnosis results are presented to the user by, for example, being displayed on the display, or being output as audio.

The output of the diagnosis result may be performed, for example, either by displaying three diagnosis results in parallel on the display, or by sequentially presenting the three diagnosis results so as to be switched. Alternatively, the first to third diagnosis operations may be presented so as to be switched, and the user specifies a diagnosis operation, thereby presenting a corresponding diagnosis result.

The user can refer to the output diagnosis results and study the results. That is, different diagnosis results are obtained from the same medical signal by different diagnosis methods, thus achieving the same effect as the second opinion, which obtains diagnosis results from a plurality of doctors.

If the diagnosis results of three diagnosis processes based on different trained models produce the same outputs, for example, the diagnosis results are considered to be reliable. On the other hand, if the diagnosis results output from three diagnosis processes are different, the user may be allowed to request reexamination or further examination.

Next, a description will be given of a second processing example of the medical information processing apparatus 10 according to the second embodiment, with reference to FIG. 9.

FIG. 9 assumes a case where the medical signal is an image, and the second processing example is an example in which a plurality of processed images acquired by the performance of slightly different image processing operations on the same trained model are taken as the input, and a plurality of diagnosis results are output.

The image is input to each of a first signal processing operation S31, a second signal processing operation S33, and a third signal processing operation S35. The image is subjected to different image processing operations by the respective signal processing functions, and three different processed images are generated.

The different image processing operations are, for example, all categorized as filtering, but are based on different approaches. Specifically, a moving average filter is run as the first signal processing operation S31, a median filter is run as the second signal processing operation S33, and a thinning operation may be performed as the third signal processing operation S35.

Through a diagnosis process that takes, as the input, three processed images obtained by different image processing operations and applies the same trained model thereto, three diagnosis results are generated. The three generated diagnosis results are displayed on the display, and thereby presented to the user.

The processing circuitry 11 may display the diagnosis results so as to be switched, either by the user's designation or automatically, in a manner similar to FIG. G. If only one of the three diagnosis results has the output “tumor is malignant”, for example, switching by the user's destination can be performed by selecting the diagnosis result “tumor is malignant” via the user's touch on a touch panel or the like, and automatic switching can be performed by selecting the minority diagnosis result. Thereafter, by executing the display control function 117, the processing circuitry 11 displays an image input to the diagnosis process which has output the selected diagnosis result, and the user becomes capable of viewing the image based on which the diagnosis result “tumor is malignant” has been made.

If the diagnosis result is clearly erroneous, for example, the user can input the correct information, and the processed medical signal and the correct information can be collected as training data, which can be also used to update the trained model.

The three diagnosis results may be aggregated, and the aggregated result may be presented to the user. For example, an aggregated result that produces a comprehensive result by majority voting may be presented, or aggregation information (a message) indicating, for example, “all three results are different”, or “two of the determination results show benignancy, and one shows malignancy” may be presented, instead of the diagnosis results themselves.

According to the second embodiment described above, a plurality of diagnosis results are presented based on a plurality of different processed medical signals acquired from a medical signal by provision of variations in the data. This allows the user to grasp the situation based on the plurality of results, and to use the output from the medical information processing apparatus according to the second embodiment as a second opinion. This results in an improvement in the user reliability.

Furthermore, the functions described in connection with the above embodiments may be implemented by, for example, installing a program for executing the processes in a computer, such as a workstation, etc., and expanding the program in a memory. The program that causes the computer to perform the technique can be stored and distributed by means of a storage medium, such as a magnetic disk (a hard disk, etc.), an optical disk (CD-ROM, DVD, Blu-ray (registered trademark) etc.), and a semiconductor memory.

The embodiments of the present invention described above are presented as examples, and do not intend to restrict the scope of the invention. The embodiments may be performed by various other forms, and may be omitted, substituted, and changed within the scope of not departing from the gist of the invention. The embodiments and their modifications are included in the scope and spirit of the invention and are included in the scope of the claimed inventions and their equivalents.

What is claimed is:

1. A medical information processing apparatus comprising processing circuitry configured to:
acquire a plurality of processed medical signals regarding a subject by performing at least one of different imaging techniques and different signal processing operations, and
output a diagnosis result by applying a trained model to the plurality of processed medical signals.

2. The apparatus according to claim 1, wherein the processing circuitry generate a processed medical signal for each signal processing operation by performing a plurality of signal processing operations as the different signal processing operations on a medical signal regarding the subject.

3. The apparatus according to claim 2, wherein the processing circuitry use a first processed medical signal generated by a first signal processing operation in a subsequent second signal processing operation, during the signal processing operations.

4. The apparatus according to claim 2, wherein the processing circuitry use the medical signal in each of the plurality of signal processing operations, during the signal processing operations.

5. The apparatus according to claim 1, wherein the processing circuitry output a single diagnosis result based on a plurality of diagnosis results.

6. The apparatus according to claim 1, wherein the processing circuitry output a single diagnosis result for each of the processed medical signals.

7. The apparatus according to claim 1, wherein the processing circuitry apply trained models including an identical parameter to the processed medical signals.

8. The apparatus according to claim 1, wherein the processing circuitry apply trained models including different parameters to the processed medical signals.

9. The apparatus according to claim 8, wherein the processing circuitry apply a trained model trained for a particular processed medical signal.

10. The apparatus according to claim 1, wherein the diagnosis result includes at least one of a result of a process of determining whether a disease is benign or malignant, a result of a process of determining whether a disease is suspected or not, and a result of a process of determining whether a treatment and/or an operation need be performed.

11. A medical information processing method, comprising:
acquiring a plurality of processed medical signals regarding a subject by performing at least one of different imaging techniques and different signal processing operations; and
outputting a diagnosis result by applying a trained model to the plurality of processed medical signals.

12. The method according to claim 11, wherein a processed medical signal is generated for each signal processing operation by performing a plurality of signal processing operations as the different signal processing operations on a medical signal regarding the subject.

13. The method according to claim 12, wherein a first processed medical signal generated by a first signal processing operation is used in a subsequent second signal processing operation, during the signal processing operations.

14. The method according to claim 12, wherein the medical signal is used in each of the plurality of signal processing operations, during the signal processing operations.

15. The method according to claim 11, wherein a single diagnosis result is output based on a plurality of diagnosis results.

16. The method according to claim 11, wherein a single diagnosis result is output for each of the processed medical signals.

17. The method according to claim 11, wherein trained models including an identical parameter are applied to the processed medical signals.

18. The method according to claim 11, wherein trained models including different parameters are applied to the processed medical signals.

19. A medical information processing system comprising:
a medical information management apparatus that stores thereon a medical signal;
a signal processing apparatus that performs a plurality of signal processing operations on the medical signal and generates a processed medical signal for each of the signal processing operations.

20. A medical information processing system according to claim 19, including a trained model trained to take the medical signal as an input, and output a diagnosis result of the medical signal, wherein
the signal processing apparatus comprises processing circuitry that outputs a diagnosis result by applying the trained model to the generated processed medical signals.
